(19) United States
(12) Patent Application Publication
(10) Pub. No.: US 2014/0087309 A1
(43) Pub. Date: Mar. 27, 2014

(54) OLEFIN-TRIGGERED ACID AMPLIFIERS

(71) Applicants: Brian Cardineau, Beaverton, OR (US); Robert L. Brainard, Albany, NY (US)

(72) Inventors: Brian Cardineau, Beaverton, OR (US); Robert L. Brainard, Albany, NY (US)

(73) Assignee: THE RESEARCH FOUNDATION OF STATE UNIVERSITY OF NEW YORK, Albany, NY (US)

(21) Appl. No.: 14/008,499

(22) PCT Filed: Mar. 28, 2012
(86) PCT No.: PCT/US2012/030840

§ 371 Date: Dec. 9, 2013

Related U.S. Application Data
(60) Provisional application No. 61/470,761, filed on Apr. 1, 2011.

(51) Int. Cl. C07C 309/73; G03F 7/20; G03F 7/004

(52) U.S. Cl. CPC C07C 309/73; G03F 7/0041; G03F 7/20
USPC 430/281.1; 558/55; 430/322

(57) ABSTRACT

There are disclosed olefinic acid amplifier triggers and methods of using these compositions in, for example, photolithography.

### CROSS-REFERENCE TO RELATED APPLICATIONS

This application claims priority from pending U.S. Provisional Patent Application 61/470,761, filed on Apr. 1, 2011, the disclosure of which is included by reference herein in its entirety.

### FIELD OF THE INVENTION

The invention relates to compositions and methods for acid amplification in photoresists and other relevant applications.

### BACKGROUND

Photolithography or optical lithography is a process used, inter alia, in semiconductor device fabrication to transfer a pattern from a photomask (sometimes called a reticle) to the surface of a substrate. Such substrates are well known in the art. For example, silicon, silicon dioxide and aluminum-aluminum oxide microelectronic wafers have been employed as substrates. Gallium arsenide, ceramic, quartz and copper substrates are also known. The substrate often includes a metal coating.

Photolithography generally involves a combination of substrate preparation, photoresist application and soft-baking, radiation exposure, development, etching and various other chemical treatments (such as application of thinning agents, edge-bead removal etc.) in repeated steps on an initially flat substrate. In some more recently-developed techniques, a hard-bake step is implemented after exposure and prior to development.

A cycle of a typical silicon lithography procedure begins by applying a layer of photoresist—a material that undergoes a chemical transformation when exposed to radiation (generally but not necessarily visible light, ultraviolet light, electron beam, or ion beam)—to the top of the substrate and drying the photoresist material in place, a step often referred to as “soft baking” of the photoresist, since typically this step is intended to eliminate residual solvents. A transparent plate, called a photomask or shadowmask, which has printed on it areas that are opaque to the radiation to be used as well as areas that are transparent to the radiation, is placed between a radiation source and the layer of photoresist. Those portions of the photoresist layer not covered by the opaque areas of the photomask are then exposed to radiation from the radiation source. Exposure is followed by development. In some cases, exposure is followed by a post-exposure bake (PEB), which precedes the development. Development is a process in which the entire photoresist layer is chemically treated. During development, the exposed and unexposed areas of photoresist undergo different chemical changes, so that one set of areas is removed and the other remains on the substrate. After development, those areas of the top layer of the substrate which are uncovered as a result of the development step are etched away. Finally, the remaining photoresist is removed by an etch or strip process, leaving exposed substrate. When a “positive” photoresist is used, the opaque areas of the photomask correspond to the areas where photoresist will remain upon developing (and hence where the topmost layer of the substrate, such as a layer of conducting metal, will remain at the end of the cycle). “Negative” photoresists result in the opposite—any area that is exposed to radiation will remain after developing, and the masked areas that are not exposed to radiation will be removed upon developing.

The need to make circuits physically smaller has steadily progressed over time, necessitating inter alia the use of light of increasingly shorter wavelengths to enable the formation of these smaller circuits. This in turn has necessitated changes in the materials used as photoresists, since in order to be useful as a photoresist, the material should not absorb light at the wavelength used. For example, phenolic materials which are commonly used for photolithography using light of wavelength 248 nm wavelength are generally not suitable for use as photoresists for light of 193 nm, since these phenolic materials tend to absorb 193 nm light.

At present, it is desired to use light in the extreme UV range (13.5 nm or shorter) for photolithography of circuits having line widths of 32-20 nm. Many of the materials which would be suitable for use as positive photoresists in this range are polymers which contain acidic groups in protected form, such as tert-butoxycarbonyl (t-BOC) protected forms of polymers derived from polyhydroxystyrene or t-butylacrylate polymers. Following the “soft bake” of the photoresist, exposure of the masked photoresist to radiation and, if necessary, post-exposure bake should result in deprotection of polymers in the areas which were not covered by the opaque portions of the mask, thus rendering these areas susceptible to attack by base, to enable the removal of these areas in the development step. In order to achieve this result, it has been proposed to utilize “chemically amplified” photoresists. The idea is to include in the photoresist an amount of a thermally stable, photolytically activated acid precursor (sometimes called a “photoacid generator” or “PAG”), so that upon irradition acid will be generated which can deprotect the irradiated portions of the positive photoresist polymer, rendering them susceptible to base attack.

In a variation on the chemical amplification technique, it has been proposed to include in the resist composition a photoacid generator, as well as an acid precursor (sometimes referred to as an “acid amplifier”) which is (a) photolytically stable and (b) thermally stable in the absence of acid but thermally active in the presence of acid. In such systems, during radiation exposure the PAG generates acid, which then during post-exposure bake acts as a catalyst to activate the acid-amplifier. Such systems are sometimes referred to in the literature as “acid amplifier” systems, since the catalytic action of the photolytically-generated acid on the second acid precursor during post-exposure bake results in an effective number of acid molecules which is higher than the number of photons absorbed during radiation exposure, thus effectively “amplifying” the effect of exposure and amplifying the amount of acid present.

Similarly, the use of PAGs and acid amplifiers in negative resists has been proposed. In these cases, the acid generated makes the areas of resist exposed to radiation less soluble in the developing solvent, usually by either effecting or catalyzing cross-linking of the resist in the exposed areas or by changing the polarity or hydrophilicity/hydrophobicity in the radiation-exposed areas of the resist.

Among the difficulties encountered in trying to implement chemical amplification photoresists systems is “outgassing”, a process whereby, as a result of acid formation, gas is generated, leading to volatile compounds that can leave the resist film while the wafer is still in the exposure tool. Outgassing can occur under ambient conditions or under vacuum as is used with extreme ultraviolet (EUV) lithography. Outgassing is a problem because the small molecules can deposit on the optics (lenses or mirrors) of the exposure tool and cause a diminution of performance. Furthermore, there is a trade-off between resolution, line-width roughness and sensitivity. A resist's resolution is typically characterized as the smallest feature the resist can print. Line width roughness is the statistical variation in the width of a line. Sensitivity is the dose of radiation required to print a specific feature on the resist, and is usually expressed in units of mJ/cm². Moreover, hitherto it has proven difficult to find acid precursors which display the requisite photostability, thermal stability in the absence of acid, and thermal acid-generating ability in the presence of acid, and which generate acids which are sufficiently strong so as to deprotect the protected resins used in photolithography.

Thus, although some acid amplifier systems have been proposed for use in photolithography using 248 nm light, there remains a need for acid amplifier systems which may be used in photolithography, particularly for use in extreme UV (13.5 nm) or electron-beam lithography.

### BRIEF DESCRIPTION OF THE INVENTION

Acid amplifiers are subdivided into components: a trigger, a body and an acid precursor. The trigger is an acid sensitive group that, when activated under acid, allows the compound to decompose and release the acid.

AAs can be classified as Generation-1, Generation-2 and Generation-3 based on the acids strength that they generate and their thermal stability. Generation-1 AAs generate weak nonfluorinated acids such as toluenesulfonic acid. Generation-2 AAs generate moderately strong fluorinated sulfonic acids such as p-(trifluoromethyl)-benzenesulfonic acid. Generation-3 AAs generate strong fluorinated sulfonic acids such as triflic acid and the AAs are thermally stable in the absence of catalytic acid. Examples of the three generations are shown below

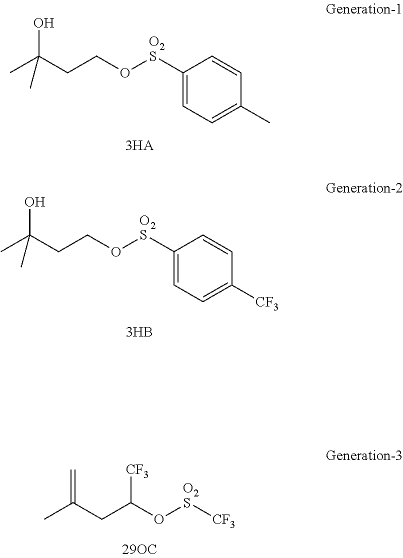

### Examples of Generation-1, Generation-2, and Generation-3 AAs

Generation 2 triggers have traditionally consisted of an acid-sensitive leaving group. Upon acidification, this group becomes protonated and causes this compound to eliminate, regenerating the original acid. The product of the elimination results in an olefin which activates the acid precursor to also eliminate. This results in a second acid being generated, and is how the acid signal is amplified, as shown below:

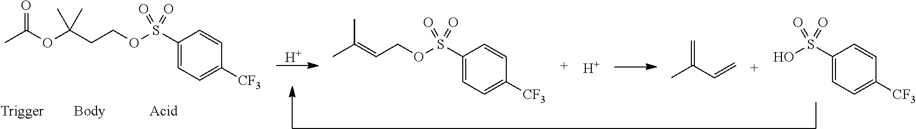

### The Acid Catalyzed Activation Mechanism for Generation II Acid Amplifiers.

Most acid amplifiers currently have triggers that are leaving groups. The acid activates the trigger; the trigger then leaves, creating a double bond. Since the double bond is allylic to the acid, the compound decomposes thermally producing an acid.

The decomposition of Generation 2 trigger types is energetically favorable in two ways. EUV photoresists utilize very strong acids (pKa˜−10). Since these triggers are generally alcohols and ethers (pKa˜−2 to −4), it is energetically favored for the acid to protonate these groups. Furthermore, the reaction of the trigger activation results in two products; the activated body-acid precursor complex and the removed trigger. This increase in the product stoichiometry is favored by entropy and thus further facilitates the trigger activation. Due to these two reasons, Generation 2 triggers can be activated very easily. However, it has been found that, for EUV photoresists, this trigger type often is too sensitive and may result in overly sensitized acid amplifiers.

Generation 3 makes use of olefin isomerization as its mechanism for activation. Under strongly acidic conditions an olefin can be acidified by a Markovnikov addition. If the acid is an adequate leaving group (such as with sulfonates), and the body is engineered properly, the olefin can isomerize from a primary carbon to a secondary or tertiary carbon. Since the olefin has moved closer to the acid precursor, the compound becomes activated, causing the acid precursor to eliminate, as shown below:

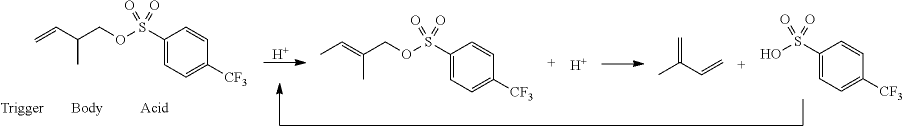

### The Acid Catalyzed Activation Mechanism for Generation 3 Acid Amplifiers.

Therefore, the current invention uses a new form of acid amplifier trigger which is activated through the isomerization of a double bond. Under acidic conditions, the double bond will isomerize from the primary carbon to the more stable secondary or tertiary carbon. Once isomerized, the double bond will then be allylic to the acid, causing the compound to decompose and the acid to be released.

It is hypothesized that Generation 3 triggers are more stable than Generation 2 triggers for two reasons. An olefin is less basic than hydroxyl or ether oxygen, and as such, it is less likely acid will protonate it. Further, the reaction of the trigger activation involves only the isomerization of one product. There is no change in entropy save for slight variations of molecular free volume.

Without being held to any one theory, applicants believe the acid amplifiers as described herein use acid-catalyzed olefin isomerization to trigger the release of acid. The olefin is positioned three carbons away from the sulfonic ester (acid precursor). In this state there is no allylic stabilization to the acid precursor and the compound is thermally stable: the trigger is effectively in the “off” position. These compounds are designed so that isomerization of the initial olefin will occur to produce the more thermodynamically favorable, more highly substituted olefin which is also allylic to the sulfonic ester. In the presence of catalytic acid, the double bond will isomerize toward the acid precursor. The compound will then thermally decompose releasing the acid, as shown below.

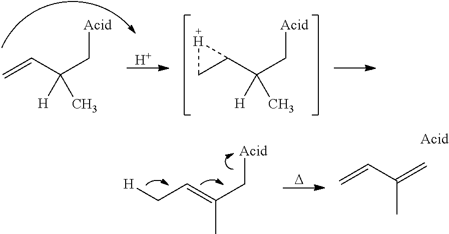

In one aspect the invention relates to a photoresist composition that includes a sulfonic acid precursor. The sulfonic acid precursor, in the presence of an acid, is capable of autocatalytically generating a sulfonic acid. In some embodiments, the sulfonic acid precursor is of formula:

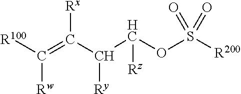

wherein

R^{w}, R^{x}, R^{y} and R^{z} are chosen independently in each instance from hydrogen, (C₁-C₈)silaalkane and (C₁-C₁₀) hydrocarbon;

R¹⁰⁰ is chosen from hydrogen and (C₁-C₂₀) hydrocarbon; or

any two of R¹⁰⁰, R^{w}, R^{x}, R^{y} and R^{z}, taken together with the carbons to which they are attached, form a (C₅-C₈) hydrocarbon ring which may be substituted with (C₁-C₈)hydrocarbon, with the proviso that the C═C double bond above is not contained within a phenyl ring;

R²⁰⁰ is chosen from
(a) —CₙHₘFₚ wherein n is 1-8, m is 0-16, p is 1-17 and the sum of m plus p is 2n+1;
(b) —CF₂CH₂OQ;
(c) —CF₂CH₂OC(═O)—R²⁰¹, wherein R²⁰¹ is selected from CH═CH₂, CCH₃═CH₂, CHQCH₂Q and CCH₃QCH₂Q; and
(d)

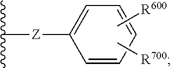

wherein Z is a direct bond, CH₂ or CF₂;

R⁶⁰⁰ is chosen from —CF₃, —OCH₃, —NO₂, F, Cl, Br, —CH₂Br, —CH═CH₂, —OCH₂CH₂Br, -Q, —CH₂-Q, —O-Q, —OCH₂CH₂-Q, —OCH₂CH₂O-Q, —CH(Q)CH₂-Q, —OC═OCH═CH₂, —OC═OCCH₃═CH₂, —OC═OCHQCH₂Q, and —OC═OCCH₃QCH₂Q;

R⁷⁰⁰ represents from one to four substituents chosen independently in each instance from H, —CF₃, —OCH₃, —CH₃, —NO₂, F, Br, and Cl; and

Q is a polymer or oligomer.

All of the compounds falling within the foregoing parent genus and its subgenera are useful for photolithography. It may be found upon examination that compounds that have been included in the claims are not patentable to the inventors in this application. In this event, subsequent exclusions of species from the compass of applicants' claims are to be considered artifacts of patent prosecution and not reflective of the inventors' concept or description of their invention; the invention encompasses all of the members of the genus described above that are not already in the possession of the public.

In one aspect the invention relates to compounds of formula

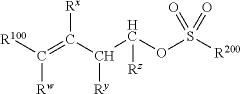

wherein

R^{w}, R^{x}, R^{y} and R^{z} are chosen independently in each instance from hydrogen, (C₁-C₈)silaalkane and (C₁-C₁₀) hydrocarbon;

R¹⁰⁰ is chosen from hydrogen and (C₁-C₂₀) hydrocarbon; or

any two of R¹⁰⁰, R^{w}, R^{x}, R^{y} and R^{z}, taken together with the carbons to which they are attached, form a (C₅-C₈) hydrocarbon ring which may be substituted with (C₁-C₈)hydrocarbon, with the proviso that the C═C double bond above is not contained within a phenyl ring;

R²⁰⁰ is chosen from
(a) —CₙHₘFₚ wherein n is 1-8, m is 0-16, p is 1-17 and the sum of m plus p is 2n+1;
(b) —CF₂CH₂OQ;
(c) —CF₂CH₂C(═O)—R²⁰¹, wherein R²⁰¹ is selected from CH═CH₂, CCH₃═CH₂, CHQCH₂Q and CCH₃QCH₂Q; and
(d)

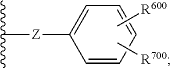

wherein Z is a direct bond, CH₂ or CF₂;

R⁶⁰⁰ is chosen from —CF₃, —OCH₃, —NO₂, F, Cl, Br, —CH₂Br, —CH═CH₂, —OCH₂CH₂Br, -Q, —CH₂-Q, —O-Q, —OCH₂CH₂-Q, —OCH₂CH₂O-Q, —CH(O)CH₂-Q, —OC═OCH═CH₂, —OC═OCCH₃═CH₂, —OC═OCHQCH₂Q, and —OC═OCCH₃QCH₂Q;

R⁷⁰⁰ represents from one to four substituents chosen independently in each instance from H, —CF₃, —OCH₃, —CH₃, —NO₂, F, Br, and Cl; and

Q is a polymer or oligomer.

In some embodiments, the invention relates to a composition for photolithography comprising a photolithographic polymer and a compound of the formula described above.

In some embodiments, the invention relates to a photoresist composition comprising a photolithographic polymer and a compound of the formula described above. In some embodiments, the photoresist composition is suitable for preparing a positive photoresist. In some embodiments, the photoresist composition is suitable for preparing a negative photoresist. In some embodiments, the photoresist composition is suitable for preparing a photoresist using 248 nm, 193 nm, 13.5 nm light, or using electron-beam or ion-beam radiation.

There is also provided, in accordance with some embodiments of the invention, a photoresist substrate which is coated with a photoresist composition in accordance with embodiments of the invention. In some embodiments, the photoresist substrate comprises a conducting layer upon which the photoresist composition is coated.

There is also provided, in accordance with embodiments of the invention, a method for preparing a substrate for photolithography, comprising coating said substrate with a photoresist composition according to embodiments of the invention.

There is also provided, in accordance with embodiments of the invention, a method for etching conducting photolithography on a substrate, comprising (a) providing a substrate, (b) coating said substrate with a photoresist composition according to embodiments of the invention, and (c) irradiating the coated substrate through a photomask.

In some embodiments, the process of coating comprises applying the photoresist composition to the substrate and baking the applied photoresist composition on the substrate.

In some embodiments, the irradiating is conducted using radiation of sufficient energy and for a sufficient duration to effect the generation of acid in the portions of the photoresist composition which has been coated on said substrate which are exposed to the radiation. For instance, said irradiation is conducted using electromagnetic radiation of wavelength 248 nm, 193 nm, 13.5 nm, or radiation from electron or ion beams.

In some embodiments, the method further comprises after the irradiating but before the developing, baking the coated substrate. In some embodiments, the baking is conducted at a temperature and for a time sufficient for the sulfonic acid precursor in the photoresist coating to generate sulfonic acid.

### DETAILED DESCRIPTION

Substituents are generally defined when introduced and retain that definition throughout the specification and in all independent claims.

In some embodiments, the invention relates to compounds of formula

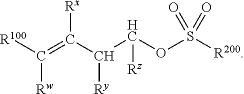

In certain embodiments, R^{w}, R^{x}, R^{y} and R^{z} are chosen independently in each instance from hydrogen, (C₁-C₈)silaalkane and (C₁-C₁₀) hydrocarbon. In some embodiments, R^{w}, R^{x}, R^{y} and R^{z} are chosen independently in each instance from hydrogen, (C₁-C₁₀)alkyl, (C₂-C₁₀)alkenyl, and a saturated or unsaturated cyclic (C₄-C₈)hydrocarbon optionally linked by a methylene. In some embodiments, R^{y} is hydrogen or (C₁-C₆)hydrocarbon. In other embodiments, R^{y} is hydrogen, methyl, ethyl or vinyl. In still other embodiments, R^{y} is selected from phenyl, alkene, or alkyne. In some embodiments, R^{x} is selected from a group that would stabilize a cation formed on the carbon to which R^{x} is attached. For instance, R^{x} may be chosen from phenyl, alkene, alkyne, cyclopropyl and —CH₂Si(CH₃)₃.

In certain embodiments, R¹⁰⁰ is chosen from hydrogen and (C₁-C₂₀) hydrocarbon. In some embodiments, R¹⁰⁰ is chosen from hydrogen, (C₁-C₁₀)alkyl, (C₂-C₁₀)alkenyl, and a saturated or unsaturated cyclic (C₄-C₈)hydrocarbon optionally linked by a methylene. In some embodiments, R¹⁰⁰ is chosen from H, methyl, ethyl, propyl, butyl and benzyl. In other embodiments, R¹⁰⁰ is chosen from H, methyl, ethyl, isopropyl, t-butyl and benzyl.

In some embodiments, any two of R¹⁰⁰, R^{w}, R^{x}, R^{y} and R^{z}, taken together with the carbons to which they are attached, form a (C₅-C₈) hydrocarbon ring which may be substituted with (C₁-C₈)hydrocarbon. In some embodiments, any two of R¹⁰⁰, R^{w}, R^{x}, R^{y} and R^{z}, taken together with the carbons to which they are attached, form a cyclopentyl or cyclohexyl ring. In some embodiments, R^{y} and R^{z} taken together form a cyclopentyl or cyclohexyl ring, each of which may be optionally substituted by (C₁-C₈)alkyl. In other embodiments, R^{x} and R^{z} taken together form a cyclopentyl or cyclohexyl ring, each of which may be optionally substituted by (C₁-C₈)alkyl.

In some aspects of the invention, the conjugation in the substituents around the C═C double bond of the skeleton can be balanced. For instance, if R¹⁰⁰ or R^{w} is an aryl group, then it would be advantageous that R^{y} should also be an aryl group. By doing so, the isomerization of the C═C double bond can occur without moving out of conjugation.

In certain embodiments, R²⁰⁰ is —CₙHₘFₚ wherein n is 1-8, m is 0-16, p is 1-17 and the sum of m plus p is 2n+1. For instance, in some embodiments, R²⁰⁰ is —CₙF₂ₙ₊₁ or —CH₂CF₃. In other embodiments, R²⁰⁰ is —CF₂CH₂OQ. In still other embodiments, R²⁰⁰ is —CF₂CH₂C(═O)—R²⁰¹, wherein R²⁰¹ is selected from CH═CH₂, CCH₃═CH₂, CHQCH₂Q and CCH₃QCH₂Q. In yet other embodiments, R²⁰⁰ is

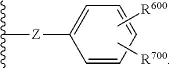

For instance, in some embodiments, R²⁰⁰ is selected from

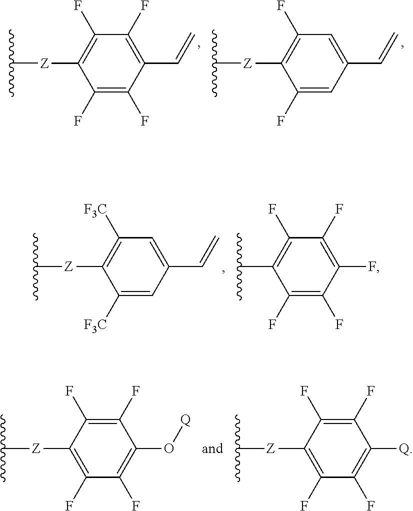

In some embodiments, Z is a direct bond. In other embodiments, Z is CH₂. In still other embodiments, Z is CF₂. In still other embodiments, Z is CHF.

In certain embodiments, R⁶⁰⁰ is chosen from —CF₃, —OCH₃, —NO₂, F, Cl, Br, —CH₂Br, —CH═CH₂, —OCH₂CH₂Br, -Q, —CH₂-Q, —O-Q, —OCH₂CH₂-Q, —OCH₂CH₂O-Q, —CH(O)CH₂-Q, —OC═OCH═CH₂, —OC═OCCH₃═CH₂, —OC═OCHQCH₂Q, and —OC═OCCH₃QCH₂Q. In certain embodiments, R⁶⁰⁰ is CF₃. In other embodiments, R⁶⁰⁰ is chosen from —CH₂Br, —CH═CH₂, and —OCH₂CH₂Br. In still other embodiments, R⁶⁰⁰ is chosen from —CH₂-Q, —O-Q, —OCH₂CH₂-Q, —OCH₂CH₂O-Q and —CH(O)CH₂-Q.

In certain embodiments, R⁷⁰⁰ represents from one to four substituents chosen independently in each instance from H, —OCH₃, —NO₂, F, Br, Cl and CᵢHⱼ(halogen)ₖ, wherein i is 1-2, j is 0-5, k is 0-5, and the sum of j plus k is 2i+1. In some embodiments, R⁷⁰⁰ represents —CF₃.

In some embodiments, Q is a polymer or an oligomer. Some suitable polymers and oligomers and the means of attachment of residues described herein to those polymers are exemplified in U.S. patent application Ser. No. 12/708,958, the relevant portions of which are incorporated herein by reference.

In some embodiments, the sulfonic acid precursor may be included in the photoresist composition as a molecule separate from the polymer. In other embodiments, the sulfonic acid precursor may be incorporated into the polymer chain. For example, if the photoresist polymer is a terpolymer having the structure

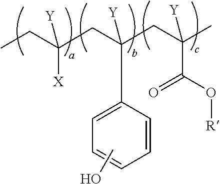

as defined in U.S. Pat. No. 6,803,169, R′ may be the sulfonic acid precursor. This can be accomplished, for example, by including in the mix of monomers used to produce the polymer an amount of a compound of formula:

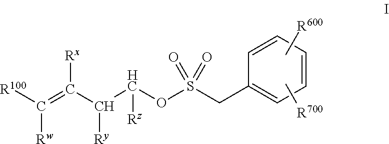

wherein R⁶⁰⁰ is chosen from —CH₂Br, —CH═CH₂, and —OCH₂CH₂Br, thus allowing the compound to be incorporated into a polymer backbone. If another acrylic acid-derived monomer containing a different group R′, e.g. tert-butyl, is also employed in the polymer synthesis, this will result in a quadpolymer rather than the terpolymer shown. Alternatively, a small amount of the quadpolymer (or terpolymer) incorporating the sulfonic acid generating compound (only) may be synthesized, and in preparing the photoresist this quad- or terpolymer may be blended with a larger amount of a terpolymer in which R′ is not a sulfonic acid generating group.

The amount of sulfonic acid precursor employed may be up to 40 mol. % of the solids of the photoresist composition, for example, between 1 and 30 mol. % of the solids of the photoresist composition, for example 2 to 20 mol. %. In the case where the sulfonic acid precursor is incorporated into the polymer, the monomer may constitute up to 40 mol. % of the polymer, for example 1 to 30% mol. % or 2 to 20% mol. %.

In some embodiments of the present invention, the photoresist composition includes a photoacid generator (PAG). PAGs are well-known in the art, see for example EP 0164248, EP 0232972, EP 717319A1, U.S. Pat. No. 4,442,197, U.S. Pat. No. 4,603,101, U.S. Pat. No. 4,624,912, U.S. Pat. No. 5,558,976, U.S. Pat. No. 5,879,856, U.S. Pat. No. 6,300,035, U.S. Pat. No. 6,803,169 and US 2003/0134227, the contents of all of which are incorporated herein by reference, and include, for example, di-(t-butylphenyl)iodonium triflate, di-(t-butylphenyl)iodonium perfluorobutanesulfonate, di-(4-tert-butylphenyl)iodonium perfluoroctanesulfonate, di-(4-t-butylphenyl)iodonium o-trifluoromethylbenzenesulfonate, di-(4-t-butylphenyl)iodonium camphorsulfonate, di-(t-butylphenyl)iodonium perfluorobenzenesulfonate, di-(t-butylphenyl)iodonium p-toluenesulfonate, triphenyl sulfonium triflate, triphenyl sulfonium perfluorobutanesulfonate, triphenyl sulfonium perfluoroctanesulfonate, triphenyl sulfonium o-trifluoromethylbenzenesulfonate, triphenyl sulfonium camphorsulfonate, triphenyl sulfonium perfluorobenzenesulfonate, triphenyl sulfonium p-toluenesulfonate, N-[(trifluoromethane sulfonyl)oxy]-5-norbornene-2,3-dicarboximide, N-[(perfluorobutane sulfonyl)oxy]-5-norbornene-2,3-dicarboximide, N-[(perfluorooctane sulfonyl)oxy]-5-norbornene-2,3-dicarboximide, N-[(o-trifluoromethylbenzene sulfonyl)oxy]-5-norbornene-2,3-dicarboximide, N-[(camphor sulfonyl)oxy]-5-norbornene-2,3-dicarboximide, N-[(perfluorobenzene sulfonyl)oxy]-5-norbornene-2,3-dicarboximide, N-[(p-toluenesulfonate sulfonyl)oxy]-5-norbornene-2,3-dicarboximide, phthalimide triflate, phthalimide perfluorobutanesulfonate, phthalimide perfluoroctanesulfonate, phthalimide o-trifluoromethylbenzenesulfonate, phthalimide camphorsulfonate, phthalimide perfluorobenzenesulfonate, phthalimide p-toluenesulfonate, diphenyl-iodonium triflate, diphenyl-iodonium perfluorobutanesulfonate, diphenyl-iodonium perfluoroctanesulfonate, diphenyl-iodonium o-trifluoromethylbenzenesulfonate, diphenyl-iodonium camphorsulfonate, diphenyl-iodonium perfluorobenzenesulfonate, diphenyl-iodonium p-toluenesulfonate. U.S. Pat. No. 6,803,169 describes the use combinations of a variety of PAGs.

Exemplary embodiments of compounds of the invention are shown below:

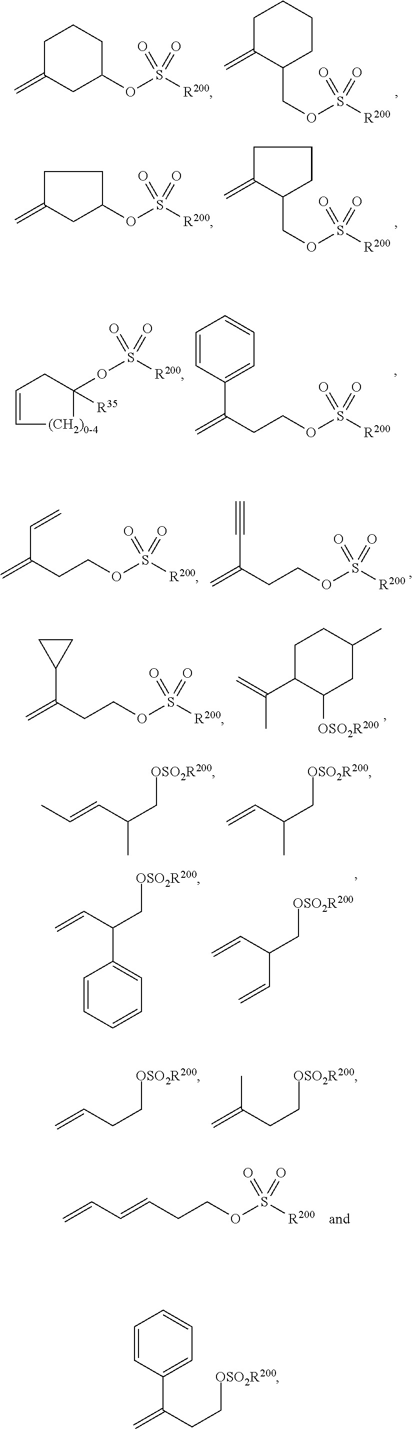

wherein R³⁵ is selected from hydrogen, (C₁-C₆)alkyl and benzyl. Compounds of the invention are not limited to those shown above; the compounds are shown merely as examples.

In the context of the present application, alkyl is intended to include linear, branched, or cyclic saturated hydrocarbon structures and combinations thereof. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

Silaalkane (or silaalkyl) refers to alkyl residues in which one or more carbons has been replaced by silicon. Examples include trimethylsilamethyl [(CH₃)₃SiCH₂—] and trimethylsilane [(CH₃)₃Si].

Hydrocarbon includes alkyl, cycloalkyl, polycycloalkyl, alkenyl, alkynyl, aryl and combinations thereof. Examples include, but are not limited to, methyl, propyl, benzyl, propargyl, vinyl, allyl, phenethyl, cyclohexylmethyl and naphthylethyl. The term “carbocycle” is intended to include ring systems consisting entirely of carbon but of any oxidation state. Thus (C₃-C₁₀)carbocycle refers to such systems as cyclopropane, benzene and cyclohexene.

The term “halogen” means fluorine, chlorine, bromine or iodine.

For purposes of this application, “polymer” and “oligomer” are as defined in U.S. patent application Ser. No. 12/708,958.

Some of the compounds described herein may contain one or more asymmetric centers and may thus give rise to enantiomers, diastereomers, and other stereoisomeric forms that may be defined, in terms of absolute stereochemistry, as (R)- or (S)-. Unless indicated otherwise, the present invention is meant to include all such possible isomers, as well as, their racemic and optically pure forms. Optically active (R)- and (S)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The configuration of any carbon-carbon double bond other than an endocyclic double bond appearing herein is selected for convenience only and is not intended to designate a particular configuration; thus a carbon-carbon double bond depicted arbitrarily herein as trans may be cis, trans, or a mixture of the two in any proportion.

Terminology related to “protecting”, “deprotecting” and “protected” functionalities occurs in some places in this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”.

The following abbreviations and terms have the indicated meanings throughout:

Ac=acetyl
BNB=4-bromomethyl-3-nitrobenzoic acid
Boc=t-butyloxy carbonyl
Bu=butyl
c-=cyclo
DBU=diazabicyclo[5.4.0]undec-7-ene
DCM=dichloromethane=methylene chloride=CH₂Cl₂
DEAD=diethyl azodicarboxylate
DIC=diisopropylcarbodiimide
DIEA=N,N-diisopropylethyl amine

### DMAP=4-N,N-dimethylaminopyridine

### DMF=N,N-dimethylformamide

DMSO=dimethyl sulfoxide
DVB=1,4-divinylbenzene
EEDQ=2-ethoxy-1-ethoxycarbonyl-1,2-dihydroquinoline
Et=ethyl
Fmoc=9-fluorenylmethoxycarbonyl
GC=gas chromatography
HATU=O-(7-Azabenzotriazol-1-yl)-1,1,3,3-tetramethyluronium hexafluorophosphate
HOAc=acetic acid
HOBt=hydroxybenzotriazole
Me=methyl
mesyl=methanesulfonyl
Ms=mesyl
MTBE=methyl t-butyl ether
NMO=N-methylmorpholine oxide
—OTf=triflate=trifluoromethanesulfonate=—OSO₂CF₃
PEB=post-exposure bake
PEG=polyethylene glycol
Ph or κ=phenyl
PhOH=phenol
PfP=pentafluorophenol
PPTS=pyridinium p-toluenesulfonate
PyBroP=bromo-tris-pyrrolidino-phosphonium hexafluorophosphate
rt=room temperature
sat'd=saturated
s-=secondary
t-=tertiary
TBDMS=t-butyldimethylsilyl
-Tf=trifyl=trifluoromethyl sulfonyl=—SO₂CF₃
triflate=—OTf=—OSO₂CF₃
TFA=trifluoroacetic acid
T_{g}=glass transition temperature
THF=tetrahydrofuran
TMOF=trimethyl orthoformate
TMS=trimethylsilyl
tosyl=Ts=p-toluenesulfonyl=—SO₂-para-(C₆H₄)—CH₃
tosylate=—OTs=—OSO₂-para-(C₆H₄)—CH₃
Trt=triphenylmethyl

A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

References herein to acid strengths or, equivalently, pKₐ values, particularly with respect to sulfonic and/or photolytically generated acids, refer to values determined by Taft parameter analysis, as such analysis is known in the art and described for example in J. Cameron et al., “Structural Effects of Photoacid Generators on Deep UV Resist Performance,” Society of Plastic Engineers, Inc. Proceedings., “Photopolymers, Principles, Processes and Mateials”, 11th International Conference, pp. 120-139 (1997) and J. P. Gutthrie, Can. J. Chem., 56:2342-2354 (1978). As reported in U.S. Pat. No. 6,803,169, HOTs (paratoluene sulfonic acid) has a pKₐ of −2.66 as determined by Taft parameter analysis. Thus, an acid which is at least as strong as HOTs will have a pKₐ of −2.66 or lower, as determined by Taft parameter analysis.

As used herein, the term “sulfonic acid precursor” refers to a molecule which can be decomposed in acidic conditions to generate HOSO₂R²⁰⁰.

As used herein, the term “photoresist polymer” refers to a polymer which may serve as the primary component in a photoresist.

As used herein, the term “photoresist substrate” refers to an article, such as a silicon wafer, which is suitable for use as a substrate in photolithography or other similar processes, and thus may have a photoresist applied thereto as part of the photolithography process.

As used herein, the term “photoresist composition” refers to a composition which may be used in connection with photolithography.

The contents of U.S. patent application Ser. No. 12/708,958 are incorporated herein by reference in their entirety. U.S. patent application Ser. No. 12/708,958 discloses, for instance (but not limited to), aspects of attachment of the acid amplifier to a polymer, appropriate polymers for use in the invention, descriptions of suitable precursors (for instance, sulfonic acid precursors), descriptions of reactions (for instance, acid catalysis) and the background of how to make and use elements of the invention in a photoresist.

It will be appreciated that because the generation of the sulfonic acid by the sulfonic acid precursor is driven, in part, by the formation of a conjugated pi-system, molecules which will not enable the formation of such systems, e.g. molecules in which the sulfonate is adjacent to a bridgehead carbon such as 2- or 7-sulfonyl norbornane, are beyond the scope of embodiments of the present invention.

### Syntheses

In general, compounds per se or for use in accordance with embodiments of present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

### Experimental

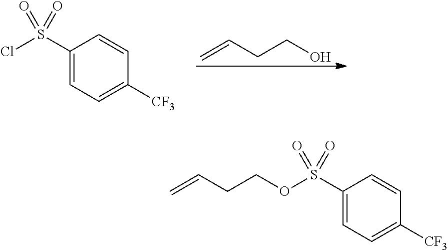

### but-3-enyl 4-(trifluoromethyl)benzenesulfonate (BC-370)

To a solution of but-3-en-1-ol (3 mmol, 0.216 g) and triethylamine (3 mmol, 0.303 g) in 10 mL of methylene chloride, 4-(α,α,α-trifluoromethyl)benzenesulfonyl chloride (2 mmol, 0.489 g) was added at 0° C. The solution was warmed to room temperature and stirred overnight. The reaction mixture was then quenched with 10 mL of 5% w/w aqueous sodium carbonate solution. The organic phase was then washed again with 10 ml of 5% w/w aqueous sodium carbonate, followed by two 10 mL of 5% w/w aqueous ammonium chloride washes and 10 mL of brine solution. The organic phase was then dried over anhydrous sodium sulfate and condensed under reduced pressure to give the desired product (0.314 g, 37%) ¹H NMR (400 MHz, CDCl₃) δ 8.03 (d, 2H, J=8 Hz), 7.81 (d, 2H, J=8 Hz), 5.66 (m, 1H), 5.09 (m, 1H), 5.06 (t, 1H, J=4 Hz), 4.14 (t, 2H, J=8 Hz), 2.42 (m, 2H).

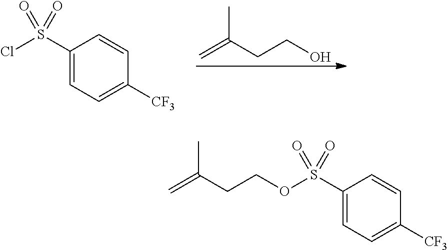

### 3-methylbut-3-enyl 4-(trifluoromethyl)benzenesulfonate (BC-371)

To a solution of 3-methylbut-3-en-1-ol (3 mmol, 0.258 g) and triethylamine (3 mmol, 0.303 g) in 10 mL of methylene chloride, 4-(α,α,α-trifluoromethyl)benzenesulfonyl chloride (2 mmol, 0.489 g) was added at 0° C. The solution was warmed to room temperature and stirred overnight. The reaction mixture was then quenched with 10 mL of 5% w/w aqueous sodium carbonate solution. The organic phase was then washed again with 10 ml of 5% w/w aqueous sodium carbonate, followed by two 10 mL of 5% w/w aqueous ammonium chloride washes and 10 mL of brine solution. The organic phase was then dried over anhydrous sodium sulfate and condensed under reduced pressure to give the desired product (0.352 g, 40%) ¹H NMR (400 MHz, CDCl₃) δ 8.03 (d, 2H, J=8 Hz), 7.81 (d, 2H, J=8 Hz), 4.80 (m, 1H), 4.68 (m, 1H), 4.21 (t, 2H, J=4 Hz), 2.37 (t, 2H, 8 Hz), 1.66 (s, 3H).

Resist Formulation

All resist solutions were made by combining ESCAP polymer (65% 4-hydroxystyrene, 25% styrene and 10% tert-butyl acrylate) with 7.5% w/w bis(tert-butylphenyl)iodonium nonaflate(PAG) and 0.5% w/w tetrabutylammonium hydroxide in 50% w/w ethyl lactate and propyleneglycol methyl ether acetate (PGMEA) to make a 5% w/w solids solution. Resists BC-370 and BC-371 were made by adding an additional 70 mmol/mL of resist, of the corresponding acid amplifier.

Lithography:

Lithography was performed at Lawrence Berkeley National Laboratories at the Berkeley microexposure tool (BMET). The three resists, BC-370, BC-371 and OS-1 (control—contains no acid amplifier) were coated on silicon wafers, baked at 120° C. for 60 seconds and exposed to extreme ultraviolet light with an open frame exposure. A series of fifty squares were exposed with incremental doses. The wafers were then baked (PEB) and developed in 0.26 N tetramethylammonium hydroxide. The wafers were then examined by light microscope and the first dose to appear completely clear (Eₒ) was observed for each wafer. This procedure was repeated for all three resists over a range of PEB temperatures. The results were recorded in the table below (Table 1).

TABLE 1
Values of dose required to clear the wafer (Eₒ)
through bake temperature (PEB) for three resists.

| | Eₒ (mJ/cm²) | | |
|---|---|---|---|
| PEB (° C.) | OS1 | BC-370 | BC-371 |
| 90 | 4.0 | 3.5 | 2.2 |
| 110 | 2.9 | 1.9 | 0.8 |
| 130 | 2.5 | 1.2 | 0.1 |

Results:

For each PEB temperature, both acid amplifiers decrease the dose required to clear the film. BC-371, in particular, shows great improvements in dose, decreasing the overall dose by about 2 mJ/cm². BC-371 has a tertiary center at one end of the olefin. It is hypothesized that this center will stabilize carbocation formation during activation and facilitate isomerization. BC-370 has a secondary carbon in place of the tertiary center. The secondary center stabilizes the carbocation much less effectively and, it is believed, should have a slower rate of isomerization and thus activation. BC-370 is slower than BC-371 to produce acid, presumably due to the slower isomerization rate of the secondary olefin.

Skilled artisans will appreciate that the alcohols may be esterified with a polymer, such as the photoresist polymer. In some cases, it is expected that this will result in higher concentrations of the acid amplifiers in the resists than would otherwise be achievable, without significant derogation from other resist properties. Furthermore, depending on the choice of acid amplifier, attachment to the polymer may be used to affect the solubility of the polymer, i.e. to create a “solubility switch”.

The invention has been described in detail with particular reference to some embodiments thereof, but it will be understood by those skilled in the art that variations and modifications can be effected within the spirit and scope of the invention.

1. A compound of formula 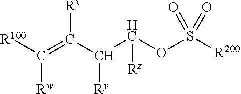
wherein
R^{w}, R^{x}, R^{y} and R^{z} are chosen independently in each instance from hydrogen, (C₁-C₈)silaalkane and (C₁-C₁₀) hydrocarbon;
R¹⁰⁰ is chosen from hydrogen and (C₁-C₂₀) hydrocarbon; or
any two of R¹⁰⁰, R^{w}, R^{x}, R^{y} and R^{z}, taken together with the carbons to which they are attached, form a (C₅-C₈) hydrocarbon ring which may be substituted with (C₁-C₈)hydrocarbon, with the proviso that the C═C double bond above is not contained within a phenyl ring;
R²⁰⁰ is chosen from
(a) —CₙHₘFₚ wherein n is 1-8, m is 0-16, p is 1-17 and the sum of m plus p is 2n+1;
(b) —CF₂CH₂OQ;
(c) —CF₂CH₂C(═O)—R²⁰¹, wherein R²⁰¹ is selected from CH═CH₂, CCH₃═CH₂, CHQCH₂Q and CCH₃QCH₂Q; and
(d) 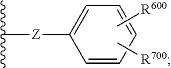
wherein Z is a direct bond, CH₂, CHF or CF₂;
R⁶⁰⁰ is chosen from —CF₃, —OCH₃, —NO₂, F, Cl, Br, —CH₂Br, —CH═CH₂, —OCH₂CH₂Br, -Q, —CH₂-Q, —O-Q, —OCH₂CH₂-Q, —OCH₂CH₂O-Q, —CH(O)CH₂-Q, —OC═OCH═CH₂, —OC═OCCH₃═CH₂, —OC═OCHQCH₂Q, and —OC═OCCH₃QCH₂Q;
R⁷⁰⁰ represents from one to four substituents chosen independently in each instance from H, —OCH₃, —NO₂, F, Br, Cl, and CᵢHⱼ(halogen)ₖ, wherein i is 1-2, j is 0-5, k is 0-5, and the sum of j plus k is 2i+1; and
Q is a polymer or oligomer.

2. A compound according to claim 1 wherein R²⁰⁰ is —CₙF₂ₙ₊₁ or —CH₂CF₃.

3. A compound according to claim 1 wherein R²⁰⁰ is

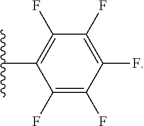 [SMILES: *c1c(F)c(F)c(F)c(F)c1F]

4. A compound according to claim 1 wherein R²⁰⁰ is

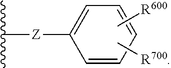

5. (canceled)

6. (canceled)

7. A compound according to claim 4 wherein R⁶⁰⁰ is CF₃.

8. A compound according to claim 4 wherein R⁶⁰⁰ is chosen from —CH₂Br, —CH═CH₂, and —OCH₂CH₂Br.

9. A compound according to claim 4 wherein R⁶⁰⁰ is chosen from —CH₂-Q, —O-Q, —OCH₂CH₂-Q, —OCH₂CH₂O-Q and —CH(O)CH₂-Q.

10. A compound according to claim 1 wherein R¹⁰⁰, R^{w}, R^{x}, R^{y} and R^{z} are chosen independently in each instance from hydrogen, (C₁-C₁₀)alkyl, (C₂-C₁₀)alkenyl, and a saturated or unsaturated cyclic (C₄-C₈)hydrocarbon optionally linked by a methylene.

11. A compound according to claim 1 wherein two of R¹⁰⁰, R^{w}, R^{x}, R^{y} and R^{z} taken together form a cyclopentyl or cyclohexyl ring.

12. A compound according to claim 1 wherein R^{y} is hydrogen or (C₁-C₆)hydrocarbon.

13. (canceled)

14. A compound according to claim 1 wherein R^{y} and R^{z} taken together form a cyclopentyl or cyclohexyl ring, each of which may be optionally substituted by (C₁-C₈)alkyl.

15. A compound according to claim 1 wherein R^{x} and R^{z} taken together form a cyclopentyl or cyclohexyl ring, each of which may be optionally substituted by (C₁-C₈)alkyl.

16. (canceled)

17. A compound according to claim 1 wherein R^{x} is selected from phenyl, alkene, alkyne, cyclopropyl and —CH₂Si(CH₃)₃.

18. A compound according to claim 1 wherein R^{y} is selected from phenyl, alkene, or alkyne.

19. A compound according to claim 1 selected from the group consisting of 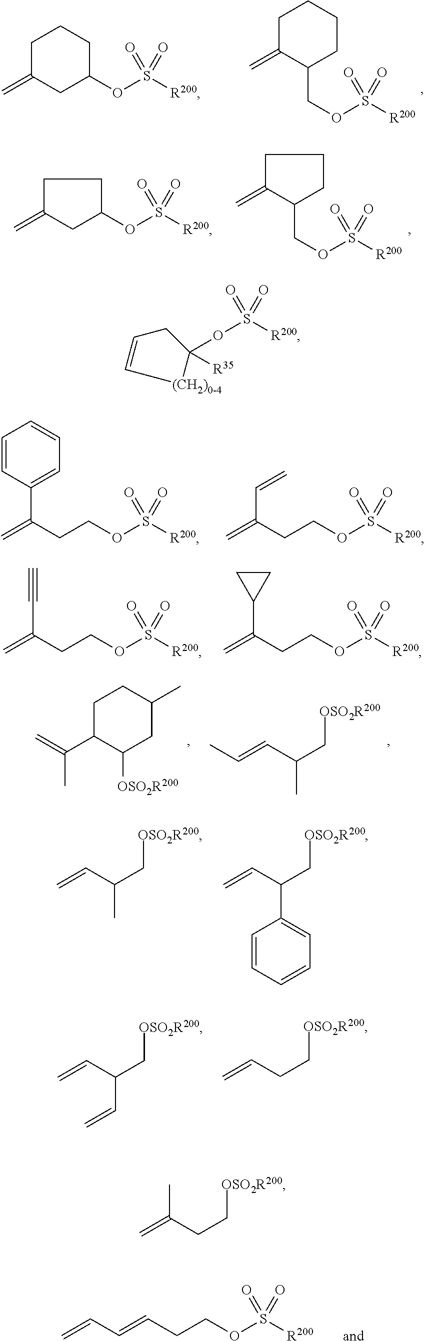 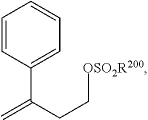
wherein R³⁵ is selected from hydrogen, (C₁-C₆)alkyl and benzyl.

20. A composition for photolithography comprising:
(a) a photolithographic polymer; and
(b) a compound according to claim 1.

21. A photoresist composition comprising:
(a) a photoresist polymer; and
(b) a compound according to claim 1.

22. A photoresist substrate which is coated with a photoresist composition according to claim 21.

23. A method for preparing a substrate for photolithography, comprising coating said substrate with a composition according to claim 21.

24. A method for conducting photolithography on a substrate, comprising (a) providing a substrate, (b) coating said substrate with a composition according to claim 21, and (c) irradiating the coated substrate through a photomask.

25. (canceled)
